(19) United States
(12) Patent Application Publication
(10) Pub. No.: US 2022/0339188 A1
(43) Pub. Date: Oct. 27, 2022

(54) ANTIMICROBIAL SHOE INSOLE AND USE THEREOF

(71) Applicants: Yuefei Tao, Wilmington, DE (US); Shuiyuan Luo, Wilmington, DE (US); Junqiu Zhu, Wilmington, DE (US); Jessica Luo, Wilmington, DE (US); Charles Tao, Wilmington, DE (US)

(72) Inventors: Yuefei Tao, Wilmington, DE (US); Shuiyuan Luo, Wilmington, DE (US); Junqiu Zhu, Quanzhou (CN); Jessica Luo, Wilmington, DE (US); Charles Tao, Wilmington, DE (US)

(21) Appl. No.: 17/811,584

(22) Filed: Jul. 10, 2022

(51) Int. Cl. A61K 33/08; A61K 33/26; A61P 31/04; A43B 7/00; A61K 33/38; A43B 17/00; A61L 2/238; A61K 33/30; A61K 33/34

(52) U.S. Cl. CPC A61K 33/08; A61K 33/26; A61P 31/04; A43B 7/00; A61K 33/38; A43B 17/006; A61L 2/238; A61K 33/30; A61K 33/34

(57) ABSTRACT

The present invention is directed to an antimicrobial shoe insole comprising an antimicrobial composition. The antimicrobial shoe insole has the density from 0.12 to 0.30 g/cm³, preferably 0.18 to 0.22 g/cm³, the hardness of the shore C is 28 to 45, the ball rebound rate is ≥65%, and anti-fatigue test (20% compression rate) is over 10,000 times. The antimicrobial shoe insole is formulated to have a sterilization rate over 99.99%, durable, and the mildew resistance index can be zero with no detectable mildew growth.

### FIELD OF DISCLOSURE

The present disclosure is directed to an antimicrobial shoe insole comprising an antimicrobial composition. The insole can be used for reducing or removing odor from shoes.

### BACKGROUND OF DISCLOSURE

The insole is a daily article commonly used in daily life. The insoles are various in variety and various in function, and comprise massage insoles, deodorant insoles and the like. The feet are easy to sweat and generate foot odor when people walk for a long time, and bacteria are easy to breed in the wet environment of the feet for a long time, so that pathological changes can be generated seriously. At present, deodorant insoles are available in the market, a deodorant layer is added on the surface of each insole, but chemical substances in the deodorant layer are easily damaged due to the friction between the soles and the deodorant layer and the mixing of sweat in the walking process, the deodorant layer is short in-service life, and the deodorant effect cannot be realized after long time.

Therefore, there is a need to provide a deodorant insole to solve the above problems.

The origin of the antibacterial material is used from ancient times, people find that water retained by silver and copper containers is not easy to deteriorate, and China has long begun to recognize that silver has an antibacterial effect. Some of antimicrobial polymers were disclosed in U.S. Pat. No. 8,858,926, filed on Mar. 31, 2011, issued on Oct. 14, 2014; U.S. Pat. No. 8,486,433, filed on pr. 29, 2005, issued on Jul. 16, 2013; U.S. Pat. No. 7,390,774, filed on Mar. 3, 2005, issued on Jun. 24, 2008. An anti-microbial composition was disclosed in a Chinese patent No.: CN109077053, filed on Jul. 14, 2018 and issued on Feb. 2, 2021.

Silver refers to a chemical substance that maintains the growth or reproduction of certain microorganisms (bacteria, fungi, yeasts, algae, viruses, etc.) below a necessary level over a period of time. Silver antimicrobials are substances or products that have bacteriostatic and bactericidal properties.

The contact reaction antibacterial mechanism is as follows: the silver ions contact and react, so that the common components of the microorganisms are damaged or dysfunction is generated. When a trace amount of silver ions reaches the microbial cell membrane, the silver ions are firmly adsorbed by virtue of coulomb attraction because the silver ions carry negative charges, penetrate through the cell wall to enter the cell and react with SH groups to solidify proteins, destroy the activity of cell synthetases, and lose division and proliferation capacity to die. Silver ions can also damage microbial electron transport systems, respiratory systems, and mass transport systems.

Because silver has strong sterilizing capability and no harm to people and livestock, more than half of airlines in the world use silver water filters at present. Swimming pools in many countries are also purified with silver, and the purified water does not irritate the eyes and skin of swimmers as it is with chemical purified water, and silver ion antibacterial agents are also applied to textile fabrics.

Antibacterial disinfection is the most commonly used technical field in daily life, medical technology and industrial fields, and the method also has various forms, such as a disinfectant method, an antibacterial method, a light irradiation method, a radiation method and the like. The silver antibacterial agent is most commonly used in the antibacterial method, the silver has strong antibacterial and bacteriostatic effects, the antibacterial effect of the silver is greatly improved due to the appearance of the nano-silver, the nano-silver has the advantages of large specific surface area, high release speed, long antibacterial time and the like, and the nano-silver is prepared by a chemical reduction method, a photo-reduction method, a radiation method and a microemulsion method, but the preparation process has high cost and pollution.

### STATEMENT OF DISCLOSURE

This disclosure is directed to an antimicrobial shoe insole comprising an upper surface and a lower surface, wherein the antimicrobial shoe insole comprises an antimicrobial composition comprising at least one metal, metal-carrying agent and a binder; wherein the metal comprises silver, silver ion, zinc, zinc ion, copper, copper ion, iron, iron ion, or a combination thereof; wherein the metal-carrying agent comprises diatomite, diatomaceous earth, montmorillonite, silica powder, oyster shell powder, shell powder, activated carbon powder, graphite powder, zinc oxide powder, aluminum oxide powder, ferric oxide powder, or a combination thereof; and wherein the metal and the metal-carrying agent are dispersed in the binder.

This disclosure is also directed to a method for reducing odor of a shoe. The method comprises placing an antimicrobial shoe insole of this disclosure to the inside of the shoe.

This disclosure is also directed to a shoe and shoe insole unit comprising the antimicrobial shoe insole of this disclosure.

### BRIEF DESCRIPTION OF DRAWING

FIG. 1A-FIG. 1B. An unlimiting example of a perspective view of the insoles. FIG. 1A: A pair of the shoe insoles includes two insoles, one for the left shoe and one for the right shoe. Each insole (1) has an upper surface (2) that can be brought into contact with a person's foot and a lower surface (3) that can be brought into contact with the inside of the shoe. Each insole has a toe end (5) and a heel end (6). FIG. 1B: Another example of a perspective view of a shoe insole.

FIG. 2A-FIG. 2B. Examples of cross-sectional views of the insole along the longitudinal axis AN of the insole. FIG. 2A: A cross-sectional view of an entire shoe insole. FIG. 2B: A cross-sectional view of a section of an insole showing multiple layers.

FIG. 3A-FIG. 3B. Examples of shoe insoles showing examples of pre-marked size marks. FIG. 3A: One example of a shoe insole. FIG. 3B Another example of a shoe insole.

FIG. 4A-FIG. 4D. Examples of microbial inhibition tests on using the antibacterial composition comprising carrier diatomaceous earth, the carrier of oyster shell powder, and silica powder. FIG. 4A and FIG. 4B: Microbial inhibition tests using Escherichia coli. FIG. 4C and FIG. 4D: Microbial inhibition tests using Staphylococcus aureus.

FIG. 5A-FIG. 5C. Examples of the topography of diatomite-loaded silver antimicrobial agent. FIG. 5A: SEM images of silver loaded diatomite at 30,000× magnification (FIG. 5A); FIG. 5B: at 20,000× magnification; and FIG. 5C: at 100,000× magnification.

FIG. 6A-FIG. 6B. Examples of the morphology of the silver-loaded antibacterial composition comprising oyster shell powder. FIG. 6A; representative SEM images of silver loaded shell powder at 20,000× magnification and FIG. 6B: at 10,000× magnification.

### DETAILED DESCRIPTION

The features and advantages of the present invention will be more readily understood, by those of ordinary skill in the art, from reading the following detailed description. It is to be appreciated that certain features of the invention, which are, for clarity, described above and below in the context of separate embodiments, may also be provided in combination in a single embodiment. Conversely, various features of the invention that are, for brevity, described in the context of a single embodiment, may also be provided separately or in any sub-combination. In addition, references in the singular may also include the plural (for example, “a” and “an” may refer to one, or one or more) unless the context specifically states otherwise.

The use of numerical values in the various ranges specified in this application, unless expressly indicated otherwise, are stated as approximations as though the minimum and maximum values within the stated ranges were both proceeded by the word “about.” In this manner, slight variations above and below the stated ranges can be used to achieve substantially the same results as values within the ranges. Also, the disclosure of these ranges is intended as a continuous range including every value between the minimum and maximum values.

The insole is a daily article commonly used in daily life. The insoles are various in variety and various in function, and comprise massage insoles, deodorant insoles and the like. The feet are easy to sweat and generate foot odor when people walk for a long time, and bacteria are easy to breed in the wet environment of the feet for a long time, so that pathological changes can be generated seriously. At present, deodorant insoles are available in the market, a deodorant layer is added on the surface of each insole, but chemical substances in the deodorant layer are easily damaged due to the friction between the soles and the deodorant layer and the mixing of sweat in the walking process, the deodorant layer is short in service life, and the deodorant effect cannot be realized after long time.

Therefore, there is a need to provide a deodorant insole to solve the above problems.

The origin of the antibacterial material is used from ancient times, people find that water retained by silver and copper containers is not easy to deteriorate, and China has long begun to recognize that silver has an antibacterial effect.

Silver refers to a chemical substance that maintains the growth or reproduction of certain microorganisms (bacteria, fungi, yeasts, algae, viruses, etc.) below a necessary level over a period of time. Silver antimicrobials are substances or products that have bacteriostatic and bactericidal properties.

The contact reaction antibacterial mechanism is as follows: the silver ions contact and react, so that the common components of the microorganisms are damaged or dysfunction is generated. When a trace amount of silver ions reaches the microbial cell membrane, the silver ions are firmly adsorbed by virtue of coulomb attraction because the silver ions carry negative charges, penetrate through the cell wall to enter the cell and react with SH groups to solidify proteins, destroy the activity of cell synthetases, and lose division and proliferation capacity to die. Silver ions can also damage microbial electron transport systems, respiratory systems, and mass transport systems.

Because silver has strong sterilizing capability and no harm to people and livestock, more than half of airlines in the world use silver water filters at present. Swimming pools in many countries are also purified with silver, and the purified water does not irritate the eyes and skin of swimmers as it is with chemical purified water, and silver ion antibacterial agents are also applied to textile fabrics.

Antibacterial disinfection is the most commonly used technical field in daily life, medical technology and industrial fields, and the method also has various forms, such as a disinfectant method, an antibacterial method, a light irradiation method, a radiation method and the like. The silver antibacterial agent is most commonly used in the antibacterial method, the silver has strong antibacterial and bacteriostatic effects, the antibacterial effect of the silver is greatly improved due to the appearance of the nano-silver, the nano-silver has the advantages of large specific surface area, high release speed, long antibacterial time and the like, and the nano-silver is prepared by a chemical reduction method, a photo-reduction method, a radiation method and a microemulsion method, but the preparation process has high cost and pollution.

In some cases, this disclosure is directed to an antimicrobial shoe insole comprising an upper surface and a lower surface, wherein the antimicrobial shoe insole comprises an antimicrobial composition comprising at least one metal, metal-carrying agent and a binder;

wherein the metal comprises silver, silver ion, zinc, zinc ion, copper, copper ion, iron, iron ion, or a combination thereof;

wherein the metal-carrying agent comprises diatomite, diatomaceous earth, montmorillonite, silica powder, oyster shell powder, shell powder, activated carbon powder, graphite powder, zinc oxide powder, aluminum oxide powder, ferric oxide powder, or a combination thereof; and

wherein the metal and the metal-carrying agent are dispersed in the binder.

In some cases, the metal-carrying agent are porous and the metal can be dispersed and supported in the porous metal-carrying agent and wherein the antimicrobial composition can be stable at high temperature. In some examples the antimicrobial composition can be stable at temperatures in a range of from 25° C. to 1000° C.

In some cases, the binder can comprise hydrogenated styrene thermoplastic elastomer, hydrogenated styrene butadiene elastomer (SEBS), chemical or physical (supercritical) foaming 3.2 SEBS, a random or block copolymer SEBS, and wherein the SEBS has a molecular weight more than 100,000 Dalton (100,000 to 500,000 Dalton), hydrogenation degree 98% or more, and a styrene content in a range of from 10% to about 40%. In prefer cases, the styrene content can be in a range of from 15% to about 25%.

In some cases, the antimicrobial shoe insole can have a density from 0.12 to 0.30 g/cm³, preferably 0.18 to 0.22 g/cm³, a hardness of the shore C value in arrange of from 28 to 45, a ball rebound rate greater than 65%, and anti-fatigue test (20% compression rate) over 10,000 times. In some cases, the ball rebound rate can be greater than 75%, greater than 85%, or greater than 90%. In some cases, the anti-fatigue test (20% compression rate) can be over 11,000 times, 15,000 times, 20,000 times, or 30,000 times. In some cases, the antimicrobial shoe insole can have a combination of the ball rebound rate being greater than 75%, greater than 85%, or greater than 90% and the anti-fatigue test (20% compression rate) being over 11,000 times, 15,000 times, 20,000 times, or 30,000 times.

In some cases, the antimicrobial composition can comprise nano-silver antibacterial agent produced with electrochemical method as disclosed above and in Chinese Patent No.: CN109077053, herein incorporated by reference.

In some cases, the antimicrobial shoe insole can be produced to have a sterilization rate at least 95-99.99%, durable for at least six months, and a mildew resistance index of zero according to the industry standard GB/T 24128-2018 (refer to Table 1). In some cases, the antimicrobial shoe insole can be produced to be durable for at least 6 months, at least 10 months, at least 12 months, at least 15 months, at least 18 months, or at least 24 months. By “durable”, it means that the antimicrobial shoe insole can maintain at least one or all of the antimicrobial properties described herein during normal wearing conditions, for a certain number of months or being washed under normal washing conditions. In some cases, the antimicrobial shoe insole can maintain a sterilization rate at least 95% for at least 6 months, at least 10 months, at least 12 months, at least 15 months, at least 18 months, or at least 24 months. In some cases, the antimicrobial shoe insole can maintain a mildew resistance index of zero for at least 6 months, at least 10 months, at least 12 months, at least 15 months, at least 18 months, or at least 24 months. In some cases, the antimicrobial shoe insole can maintain a sterilization rate at least 95% and a mildew resistance index of zero for at least 6 months, at least 10 months, at least 12 months, at least 15 months, at least 18 months, or at least 24 months. In some cases, the antimicrobial shoe insole can be washed under normal washing conditions, such as with water, soap or detergents, for multiple times, such as more than 50 times, more than 100 times, more than 150 times, or more than 200 times.

TABLE 1
Mildew resistance index according to GB/T 24128-2018.

| Resistance | | |
|---|---|---|
| index | Mildew growth | Description |
| 0 | No growth | The material is resistant to mold attack |
| 1 | Primary growth | The material is partly resistant to mold |
| | | attack or generally less susceptible to |
| | | mold attack |
| 2 | Visible growth | The material is susceptible to mold |
| | and sporulation | attack |

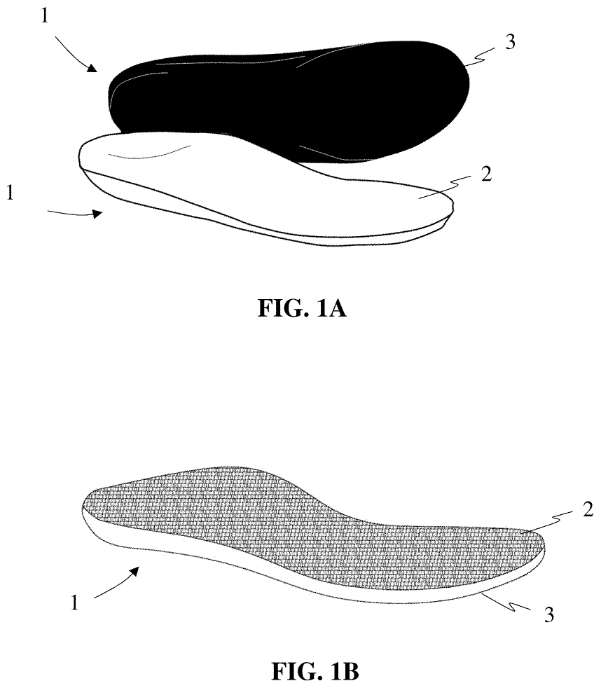

Some unlimited examples of the antimicrobial shoe insole (1) can be shown in FIG. 1A and FIG. 1B having an upper surface (2) and a lower surface (3).

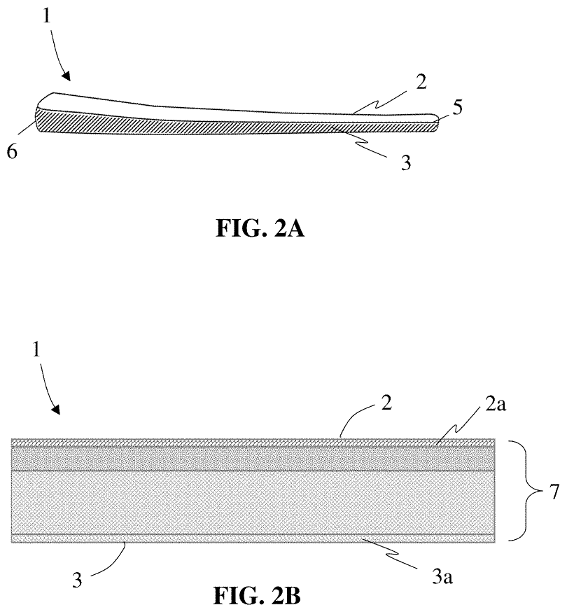

An unlimited example is shown schematically in FIG. 2A for a cross sectional view of the antimicrobial shoe insole (1) showing the upper surface (2) and lower surface (3). The upper surface (2) that can be brought into contact with a person's foot and a lower surface (3) that can be brought into contact with the inside of a shoe. Each antimicrobial shoe insole can have a toe end (5) and a heel end (6). In some cases, the upper surface (2) can be the surface of an upper layer (2a) that is exposed to the exterior of the antimicrobial shoe insole. The lower surface (3) can be the surface of a lower layer (3a) that is exposed to the exterior of the antimicrobial shoe insole. In some cases, the upper layer (2a) and the lower layer (3a) can be bound together forming the antimicrobial shoe insole (1) (FIG. 2A). The upper layer, the lower layer, or a combination thereof, can comprise the antimicrobial composition disclosed herein.

In some cases, the antimicrobial shoe insole can further comprise at least one intermediate layer (7) positioned between the upper surface and lower surface (FIG. 2B). In some cases, the antimicrobial shoe insole can comprise one intermediate layer. In some cases, the antimicrobial shoe insole can comprise two intermediate layers. In some cases, the antimicrobial shoe insole can comprise three intermediate layers. In some cases, the antimicrobial shoe insole can comprise four intermediate layers.

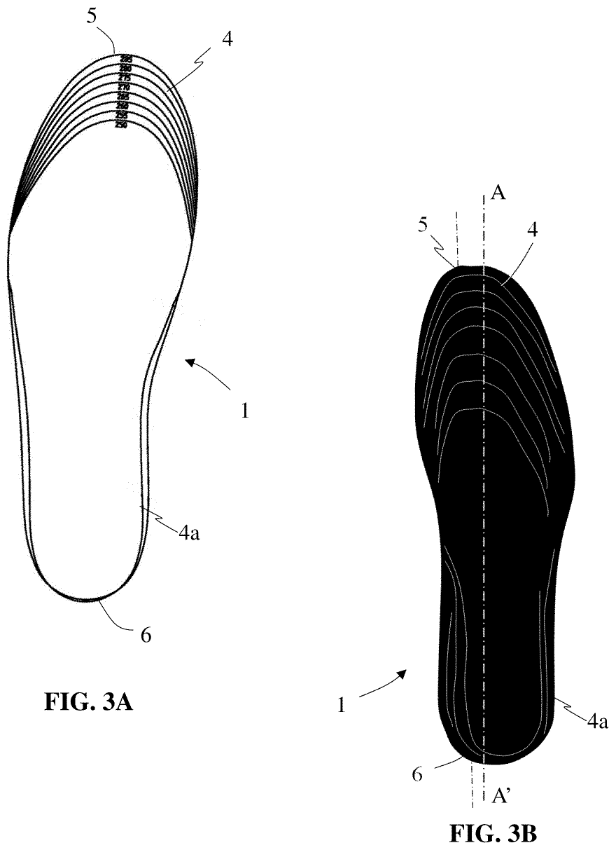

In some cases, the antimicrobial shoe insole can further comprise one or more pre-marked size markers on the upper surface, the lower surface, or a combination thereof. In some cases, the antimicrobial shoe insole can comprise pre-marked size markers that can comprise toe end pre-marked size markers (4) near or at the toe end and heel end pre-marked size markers (4a) near or at the heel end, such as those shown in FIG. 3A and FIG. 3B. The antimicrobial shoe insole can have a longitudinal axis A-A′ of the insole extending from the toe end (5) to the heel end (6) across the insole (FIG. 3B). In some cases, FIG. 3A can be a representative schematic top-down view of an insole showing an unlimited example of multiple toe end pre-marked size markers (4) and one heel end pre-marked size markers (4a). In some cases, FIG. 3B can be a representative schematic bottom-up view of an insole showing an unlimited example of multiple toe end pre-marked size markers (4) and heel end pre-marked size markers (4a).

In some cases, about 10% to 100% of the antimicrobial composition can be positioned on the upper surface. In some cases, about 10% to 100% of the antimicrobial composition can be positioned in the upper layer and can be available on the upper surface. In some cases, about 10% to 100% of the antimicrobial composition are positioned on the lower surface. In some cases, about 10% to 100% of the antimicrobial composition can be positioned in the lower layer and can be available on the lower surface. In some cases, about 10% to 100% of the antimicrobial composition can be positioned in the lower layer and can be available on the lower surface and in the lower layer and can be available on the lower surface, while about 0% to about 10% of the antimicrobial composition can be positioned in the intermediate layers. All percentages are based on the total weight of the antimicrobial composition in each antimicrobial shoe insole.

In some cases, the antimicrobial shoe insole can comprise about 70% to 100%, 80% to 100%, 90% to 100% or 100% of the antimicrobial composition in the upper layer and can be available on the upper surface, all percentages based on the total weight of the antimicrobial composition in each antimicrobial shoe insole.

In some cases, the antimicrobial shoe insole can further comprise at least one intermediate layer positioned between the upper surface and lower surface, wherein the intermediate layer comprises moisture absorbing materials. In some cases, the intermediate layer further comprises the antimicrobial composition. Moisture absorbing materials known in the industry can be suitable.

In some cases, the antimicrobial shoe insole of this disclosure can comprise the antimicrobial composition that comprises a cycle of nano-silver to silver ion to nano-silver producing durable antimicrobial property. In some cases, nano-silver particles on the surface of diatomite can easily generate silver ions when in contact with water, such as sweat from feet. The silver ions can enter bacteria cells and interfere with the normal metabolism of bacteria, achieve the purpose of antimicrobial function. When bacteria cells die, silver ions can be release and reduced from monovalent to the silver element. The silver element then can be deposited back on to the surface by the adsorption of the diatomite in the insole, completing a cycle of nano-silver to silver ion to nano-silver at the surface of the antimicrobial shoe insole.

In some cases, the antimicrobial shoe insole of this disclosure can comprise in a range of from 1% to 99% of silver element and 99% to 1% of monovalent silver ion, percentage based on the total weight of silver in the antimicrobial shoe insole. In some cases, the antimicrobial shoe insole of this disclosure can comprise in a range of from 1% to 99%, 5% to 99%, 10% to 99%, 15% to 99%, 20% to 99%, 25% to 99%, 30% to 99%, 35% to 99%, 40% to 99%, 45% to 99%, 50% to 99%, 55% to 99%, 60% to 99%, 65% to 99%, 70% to 99%, 75% to 99%, 80% to 99%, 85% to 99%, 90% to 99%, or 95% to 99% of nano-silver dispersed in the metal-carrying agent and the binder disclosed herein, percentage based on the total weight of silver in the antimicrobial composition in the antimicrobial shoe insole. In some cases, the metal-carrying agent comprises diatomite, diatomaceous earth, montmorillonite, silica powder, oyster shell powder, shell powder, activated carbon powder, graphite powder, zinc oxide powder, aluminum oxide powder, ferric oxide powder, or a combination thereof. In some cases, the binder can comprise hydrogenated styrene thermoplastic elastomer, hydrogenated styrene butadiene elastomer (SEBS).

This disclosure is also directed to a method for reducing odor of a shoe. The method can comprise placing an antimicrobial shoe insole of this disclosure to the inside of the shoe. Any of the antimicrobial shoe insole disclosed herein can be suitable. The shoe can be any shoe. In some cases, the shoe can be a sprots shoe, a dress shoe, a walking shoe, a running shoe, or a leisure shoe.

This disclosure is also directed to a shoe and shoe insole unit comprising the antimicrobial shoe insole of this disclosure. Any of the antimicrobial shoe insole disclosed herein can be suitable. Any of the shoes disclosed herein can be suitable. The shoe and shoe insole unit can comprise a pair of shoes each having one antimicrobial shoe insole of this disclosure placed within.

The antimicrobial shoe insole disclosed here can have an advantage of having high-performance comfort. The insole of the present disclosure can comprise hydrogenated styrene thermoplastic elastomer. When the degree of hydrogenation of the elastomer is higher than 98%, the molecular weight can be higher than 100,000 g/mol, and the styrene content can be lower than 40% (especially lower than 20%), the composite antibacterial insole based on this elastomer has high resilience, low hardness (soft) and low density (light), and excellent fatigue properties.

Another advantage of the antimicrobial shoe insole of this disclosure can be environmental-benefits: Compared with the traditional preparation process of nano-silver antibacterial agent, the antibacterial agent used in the present invention is prepared by electrochemical method, and has zero emission, zero pollution, environmental-friendly, and low price. Moreover, nano-silver loaded on porous diatomite has the following advantages: 1. High release efficiency of silver ions; 2. No dyeing of the prepared materials, which is conducive to further processing into various colors; 3. Antibacterial at the same time with water-absorbing properties, which can not only kill bacteria contaminated on the surface of the material, but also inhibit the formation of biofilms on the surface of the material.

Yet another advantage of the antimicrobial shoe insole of this disclosure can have the cycle of nano-silver to silver ion to nano-silver producing efficient and durable antibacterial property. The antibacterial composition of the present disclosure is a porous hydrophilic inorganic material. In some cases, the antibacterial composition can comprise diatomite-loaded nano-silver composite comprising nano-silver particles prepared by an electrochemical method. The nano-silver particles on the surface of diatomite can easily generate silver ions when in contact with water. The positive charge of silver ions can inhibit the transport of nutrients through the bacterial cell membrane that is typically negatively charged. Secondly, silver ions can also enter the cells of bacteria and interfere with the normal metabolism of bacteria. achieve the purpose of sterilization. When the reducing substances secreted when bacteria died, silver ions can be reduced from monovalent to silver elements, and then silver elements can be deposited on the surface by the adsorption of water on diatomite. In this way, the cycle of nano-silver to silver ion to nano-silver enables the nano-silver/diatomite composite material to have an efficient, durable and stable antibacterial effect. In addition, the nano-silver/diatomite composite material can be fully and effectively dispersed and mixed in the hydrogenated styrene-based functional polymer material, and micropores or holes in the microscopic state can be formed inside the material. The nano-silver/diatomite composite material can easily migrate from the interior of the styrene-based functional polymer material to the capillary-like channel on its surface, so that it has efficient and durable antibacterial properties. In some cases, the antimicrobial shoe insole of this disclosure can be durable for use in normal wearing conditions for over 12 months.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### Detailed Description Procedures

Preparation method: add a mixture of powder materials including diatomite, montmorillonite, silica powder, oyster shell powder, activated carbon powder, graphite powder, zinc oxide powder, aluminum oxide powder or ferric oxide powder into water and vigorously stir to form a turbid suspension solution. Use this cloudy solution as the electrolyte. With pure silver as the anode, the shape can be rod-shaped, sheet-shaped or other shapes; the cathode can be made of metallic zinc or titanium electrode. Direct current is passed between the cathode and the anode. The electrolyte is continuously stirred during the electrolysis process.

After the electrolysis, the silver-carrying powder material in the electrolyte is filtered out, then dried in a vacuum drying oven, and the fully dried silver-loaded powder material is put into a corundum crucible and heat-treated in a muffle furnace at 250-800° C. for 0.5˜8 h, after Ag₂O and AgOH are thermally decomposed into elemental silver, and then cooled to room temperature naturally, the silver-loaded antibacterial agent is obtained, and the size of silver particles is 10 nm˜5 μm.

The technical solution of the present invention is further illustrated by the following examples.

### Example 1: Diatomite Silver-Loaded Antibacterial Agent, the Silver Loading is about ˜5%

Preparation process: using diatomite as a carrier, the diatomite-silver-loaded antibacterial material is prepared by an electrolytic method. Add 100 g of diatomite original soil into a 1.5 L glass bottle with 1.1 L distilled water and stir well. Adjust the rotating speed of the electronic stirrer to 300-500 rpm, keep stirring continuously, use metallic silver as the anode, metallic zinc as the cathode, the current is 0.35 A, and the electrolysis is carried out for 5 h. After electrolysis, filtration is performed, and the wet sample is dried in an oven at 70-100° C., and then heat-treated at 400-450° C. for 1 hour to obtain diatomite-silver-loaded antibacterial material.

### Example 2: Diatomite Silver/Zinc Antibacterial Agent, the Silver Loading is about 2.5%, and the Zinc Loading is about ˜2.5%

Preparation process: using diatomite as a carrier, the diatomite-silver-loaded antibacterial material is prepared by an electrolytic method. Weigh 100 g of diatomite original soil into a 1.5 L glass bottle, add 1.1 L distilled water, stir well. Adjust the rotation speed of the electronic stirrer to 300-500 rpm, and keep stirring; take the metal silver wire as the anode, the metal zinc wire as the cathode, the current is 0.35 A, and electrolyze for 2 h; then use the metal zinc as the anode and the metal silver as the cathode, the current is 0.6 A, and the electrolysis is performed for 5 hours; after the electrolysis is completed, filtration is performed, and the wet sample is dried in an oven at 70° C.-100° C., and then heat-treated at 400-450° C. for 1 hour to obtain diatomite-silver/zinc antibacterial material.

### Example 3: Oyster Shell Powder Silver/Zinc/Copper Antibacterial Agent, the Silver Loading is about ˜1%, the Zinc Loading is about ˜2.5%, and the Copper Loading is about ˜1.5%

Preparation process: using diatomite as a carrier, the diatomite-silver-loaded antibacterial material is prepared by an electrolytic method. Weigh 100 g of diatomite original soil into a 1.5 L glass bottle, add 1.1 L distilled water, stir well. Adjust the rotating speed of the electronic stirrer to 300-500 rpm, and keep stirring; take the metal silver wire as the anode, the metal zinc wire as the cathode, the current is 0.35 A, and electrolyze for 1 h; then use the metal zinc as the anode and the metal silver as the cathode. The current is 0.6 A, and electrolysis is performed for 5 hours; then metal copper is used as the anode, metal silver is used as the cathode, and the current is 0.6 A, and the electrolysis is performed for 3 hours; Then heat treatment at 400-450° C. for 1 hour to prepare diatomite-silver/zinc/copper antibacterial material.

### Example 4: Silica Powder Carries Silver/Zinc/Iron Antibacterial Agent, the Silver Loading is about ˜3%, the Zinc Loading is about ˜2.5%, and the Iron Loading is about ˜0.5%

Preparation process: using diatomite as a carrier, the diatomite-silver-loaded antibacterial material is prepared by an electrolytic method. Weigh 100 g of diatomite original soil into a 1.5 L glass bottle, add 1.1 L distilled water, stir well. Adjust the rotating speed of the electronic stirrer to 300-500 rpm, and keep stirring; take the metal silver wire as the anode, the metal zinc wire as the cathode, the current is 0.35 A, and electrolyze for 3 hours; then use the metal zinc as the anode and the metal silver as the cathode. The current is 0.6 A, and the electrolysis is performed for 5 h; then metal iron is used as the anode, metal silver is used as the cathode, the current is 0.5 A, and the electrolysis is performed for 1.5 h. The diatomite-silver/zinc/iron antibacterial material can be prepared by heat treatment at 400-450° C. for 1 hour.

### Comparative Examples

Comparative Example 1: Diatomaceous earth (commercially available).

Comparative Example 2: Oyster shell powder (commercially available).

Comparative Example 3: Silica powder (commercially available).

Comparative Example 4: Nano-zinc oxide (commercially available).

### Microbial Inhibition Zone Tests

Escherichia coli or Staphylococcus aureus were used as test strains. Antibacterial effects of inorganic antibacterial materials were analyzed by the zone of inhibition method. The microbial inhibition tests were conducted as described below:

(1) All items used in the experiment (such as petri dishes, pipette tips, test tubes, and inorganic antibacterial materials, etc.) were sterilized with autoclave or UV irradiation;

(2) Agar medium for proper microbial strain were prepared and pour it into a petri dish forming test plates;

(3) Evenly spread bacterial culture liquid, Escherichia coli or Staphylococcus aureus, on the surface of test plates. Place antibacterial material samples from Examples 1-4 (Exp 1-Exp 4) and Comparative Examples 1-4 (Com 1-Com 4) on the surface of the test plate, and incubated at 37° C. for 24 hours; and

(4) After 24 hours, diameters of the bacteriostatic rings, also referred to as Inhibition Zones around the antibacterial material samples were measured and photographed.

The antibacterial agent and the sample (powder) of the comparative example were pressed into pieces with a diameter of 4 mm After autoclave sterilization, the samples were replicated onto agar petri dish test plate incubated for 24 hours.

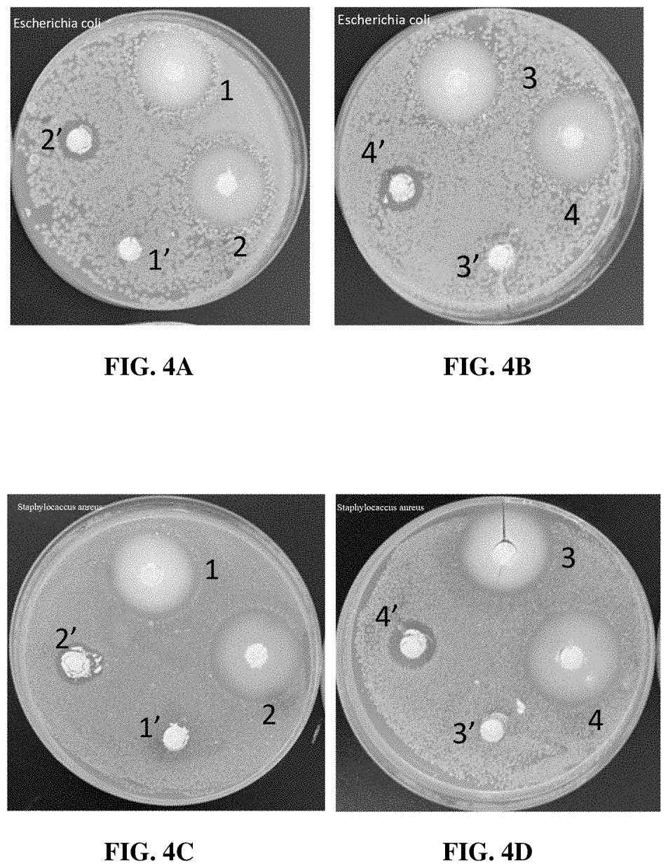

Representative microbial inhibition test results are shown in Table 2, Table 3 and FIG. 4A-FIG. 4D. Samples from Exp 1 (shown as 1), Exp 2 (shown as 2), Com 1 (shown as 1′) and Com 2 (shown as 2′) were tested on an Escherichia coli test plate (FIG. 4A) and Staphylococcus aureus test plate (FIG. 4C). Samples from Exp 3 (shown as 3), Exp 4 (shown as 4), Com 3 (shown as 3′) and Com 4 (shown as 4′) were tested on an Escherichia coli test plate (FIG. 4B) and Staphylococcus aureus test plate (FIG. 4D).

TABLE 2
Microbial Inhibition Test with Escherichia coli.

| | Exp | Exp | Exp | Exp | Com | Com | Com | Com |
|---|---|---|---|---|---|---|---|---|
| | 1 | 2 | 3 | 4 | 1 | 2 | 3 | 4 |
| Inhibition | 11.4 | 12.2 | 11.3 | 12.3 | N/A | 6.1 | N/A | 7.1 |
| zone | | | | | | | | |
| diameter | | | | | | | | |
| (mm) | | | | | | | | |

N/A: Measurement on the ring diameter was not available indicating no inhibition of microbial growth.

TABLE 3
Microbial Inhibition Test with Staphylococcus aureus.

| | Exp | Exp | Exp | Exp | Com | Com | Com | Com |
|---|---|---|---|---|---|---|---|---|
| | 1 | 2 | 3 | 4 | 1 | 2 | 3 | 4 |
| Inhibition | 14.1 | 13.1 | 14.9 | 14.0 | N/A | 5.2 | N/A | 7.0 |
| zone | | | | | | | | |
| diameter | | | | | | | | |
| (mm) | | | | | | | | |

N/A: Measurement on the ring diameter was not available indicating no inhibition of microbial growth.

### Microbial Viability Tests:

Foamed sheets containing antimicrobial compositions prepared in Examples 1-4 (Exp 1-Exp 4) were washed for 100 times and then cut into test pieces of about 5 cm×5 cm in size.

Microbial cultures of Escherichia coli, Klebsiella pneumoniae, Candida albicans were prepared according to concentrations specified in Table 4.

Microbial strains Staphylocaccus anreus (ATCC 6538), Klebsiella pneumoniae (ATCC 4352) and Candida albicans (ATCC 10231) were each grown to confluence. Aliquots of microbial culture of each of the strains were coated on the surface of a sterilized test piece containing one of the antimicrobial compositions, and then covered with a layer of sterile film.

The test pieces coated with microbial culture were incubated for 24 hours at 37° C. for Escherichia coli, Klebsiella pneumoniae and at 37° C. for Candida albicans.

The test pieces were then each placed into an Erlenmeyer flask containing proper liquid growth nutrient medium for each of the microbials and shake for 24 hours.

A predetermined volume of the culture from each of the Erlenmeyer flasks was spread evenly on the surface of agar plate with proper growth nutrient medium for each of the microbials and incubated for 24 h.

The number of visible microbial colonies (colony forming units, CFU/piece) on each of the agar plates was counted and photographed. Results are shown in Table 4.

TABLE 4
Antibacterial test results.

| | | | The number | The number | The number | |
|---|---|---|---|---|---|---|
| | | | of viable | of viable | of viable | |
| | | | bacteria | bacteria | bacteria | |
| | | | colonies at | colonies at | colonies at | |
| | | | Time 0 | Time 24 h | Time 24 | |
| | Action time | | (Blank Control) | (Blank Control) | (Sample) | Inhibition |
| | microorganism | Action time | (CFU/piece) | (CFU/piece) | (CFU/piece) | Rate (%) |
| Exp 1 | Staphylococcus | 24 h | 3.4 × 10⁵ | 1.2 × 10⁷ | <20 | >99.9 |
| | aureus | | | | | |
| | Klebsiella | 24 h | 3.8 × 10⁵ | 1.2 × 10⁶ | <20 | >99.9 |
| | pneumoniae | | | | | |
| | Candida | 48 h | 1.6 × 10⁵ | 1.0 × 10⁶ | <20 | >99.9 |
| | albicans | | | | | |
| Exp 2 | Staphylococcus | 24 h | 3.4 × 10⁵ | 1.2 × 10⁷ | <20 | >99.9 |
| | aureus | | | | | |
| | Klebsiella | 24 h | 3.8 × 10⁵ | 1.2 × 10⁶ | <20 | >99.9 |
| | pneumoniae | | | | | |
| | Candida | 48 h | 1.6 × 10⁵ | 1.0 × 10⁶ | <20 | >99.9 |
| | albicans | | | | | |
| Exp 3 | Staphylococcus | 24 h | 3.4 × 10⁵ | 1.2 × 10⁷ | <20 | >99.9 |
| | aureus | | | | | |
| | Klebsiella | 24 h | 3.8 × 10⁵ | 1.2 × 10⁶ | <20 | >99.99 |
| | pneumoniae | | | | | |
| | Candida | 48 h | 1.6 × 10⁵ | 1.0 × 10⁶ | <20 | >99.99 |
| | albicans | | | | | |
| Exp 4 | Staphylococcus | 24 h | 3.4 × 10⁵ | 1.2 × 10⁷ | <20 | >99.99 |
| | aureus | | | | | |
| | Klebsiella | 24 h | 3.8 × 10⁵ | 1.2 × 10⁶ | <20 | >99.99 |
| | pneumoniae | | | | | |
| | Candida | 48 h | 1.6 × 10⁵ | 1.0 × 10⁶ | <20 | >99.99 |
| | albicans | | | | | |

### Silver-Loaded Antibacterial Material:

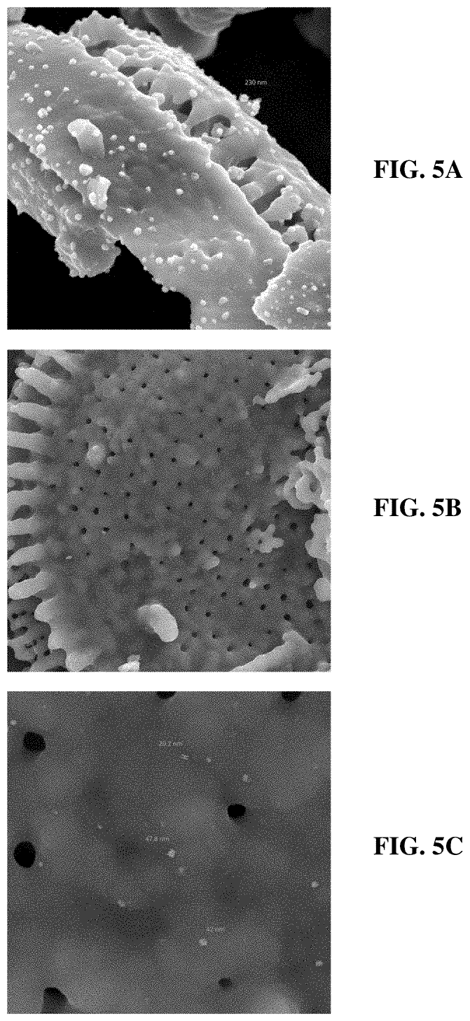

About 100 g of diatomite original soil was added into a 1.5 L glass bottle with 1.1 L of distilled water and stirred well. The speed of the electronic stirrer was adjusted to 300-500 rpm. Electrolysis was performed for 5 h with metallic silver as the anode, metallic zinc as the cathode, and electric current at 0.35 A. After the electrolysis was completed, the silver-carrying powder material in the electrolyte is filtered out. The silver-carrying powder material was dried in an oven at 70° C.-100° C., and then heat-treated at 400-450° C. for 1 hour to obtain a diatomite-silver-loaded antibacterial material. Scanning Electron Microscopy (SEM) was conducted. FIG. 5 shows representative SEM images of silver loaded diatomite at 30,000× magnification (FIG. 5A), 20,000× magnification (FIG. 5B) and 100,000× magnification (FIG. 5C).

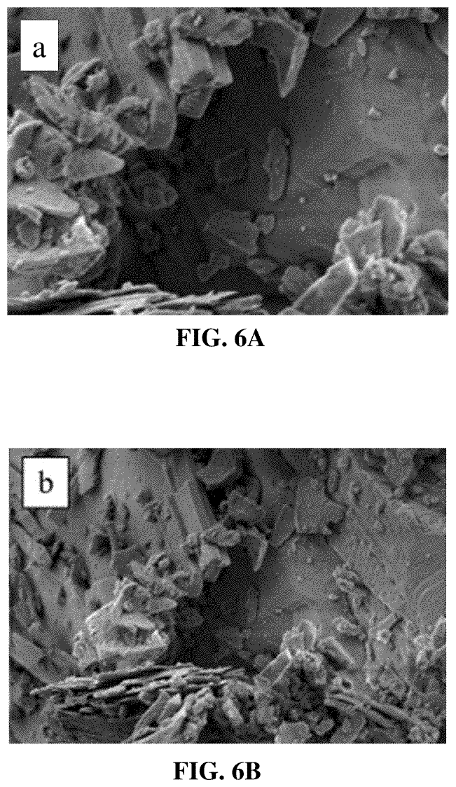

About 100 g of oyster shell powder was added into a 1.5 L glass bottle with 1.1 L of distilled water and stirred evenly. The rotation speed of the electronic stirrer was adjusted to 300-500 rpm, continue stirring. Electrolysis was performed for 1 h using metallic silver as the anode, metallic zinc as the cathode, and electric current at 0.35 A. Filter using any known solid/liquid filtration media and process after electrolysis to obtain a wet intermediate. The wet intermediate was dried in an oven at 70° C.-100° C., and then heat-treated at 400-450° C. for 1 hour to obtain oyster shell powder-silver-loaded antibacterial material. FIG. 6 shows representative SEM images of silver loaded shell powder at 20,000× magnification (FIG. 6A) and 10,000× magnification (FIG. 6B).

### Antimicrobial Shoe Insole Manufacturing Process:

Formula materials containing SEBS, antibacterial agent, foaming agent, cross-linking agent and stearic acid are batched into a mixer according to the proportion in Table 5. The formula materials were mixed in the mixer for 7 to 9 minutes at a temperature of about 110 to 120° C. The mixed materials were then poured into a granulator for granulation forming pellets. The pellets were poured into a mold at a mold temperature of about 160-180° C. for a foaming time of about 5-10 minutes to obtain a foamed sheet. The foamed sheet was punched and cut into a predefined shape. A layer of cloth was hot-pressed, shaped and cut to produce finished product antibacterial shoe insole.

In one example, the cloth mold was hot-pressed onto the foamed sheet using a hot-press mold that has a set of scale lines. During hot-pressing, the scale lines were reverse-printed on the back of the shoe insole to form size scale lines (FIG. 3A and FIG. 3B).

In another example, size lines were printed onto the underside of shoe insole. The finished shoe insoles were in the same shape and look as those produced using hot-press mold shown in FIG. 3A and FIG. 3B.

Among them, Example 5 used the antibacterial composition of Example 1; Example 6 used the antibacterial composition of Example 2; Example 7 used the antibacterial composition of Example 3; and Example 8 used the antibacterial composition of Example 4.

### Shoe Insole Physical Performance Test Standard:

Hardness (Shore C) was tested with a shore C hardness tester according to GB/T531-2008.

Rebound (%) The test was carried out according to GB/T6670-2008 standard using a falling ball rebound tester.

Compression deformation/skew (%) measurement was conducted according to GB HG/T 2876-2009.

In brief, SEBS insole samples were manufactured according to the formula in Table 5.

Test Procedure:

1. Number the samples and measure the geometric dimensions of the samples.
2. The length and width of the specimen are measured with vernier calipers, accurate to 0.02 mm.
3. The height of the sample before and after compression is measured with a thickness gauge, accurate to 0.01 mm.
4. There are no less than 3 samples for each sample, and the height difference of the same group of samples is no less than 0.1 mm.
5. The formed insole was placed between compression plates and kept in a constant temperature of 50° C. for the about 72 h (±2 h). The shoe insole samples were removed from the compression plates, laid open for 2 hours (parking). Measurements were taken. Data are shown in Table 6.

Test Results:

K=(H₀−H)/H₀×100

In the formula: K—compression deformation, the unit is %;

H₀—the height of the sample before the test, the unit is mm;

H—The height of the sample after parking after the test, the unit is mm.

Take the arithmetic mean of the three samples as the test result, accurate to one decimal place.

TABLE 5
Foaming formula of SEBS insole (weight parts).

| | Example 5 | | Example 6 | | Example 7 | | Example 8 | |
|---|---|---|---|---|---|---|---|---|
| Anti- | 1 part of | | 1 part of | | 1 part of | | 1 part of | |
| bacterial | that of | | that of | | that of | | that of | |
| Composition | Example 1 | | Example 2 | | Example 3 | | Example 4 | |
| SEBS | 100 | parts | 100 | parts | 100 | parts | 100 | parts |
| foaming | 3.0 | parts | 3.0 | parts | 3.0 | parts | 3.0 | parts |
| agent | | | | | | | | |
| cross-linking | 1.0 | parts | 1.0 | parts | 0.8 | parts | 0.8 | parts |
| agent | | | | | | | | |
| Stearic acid | 1.5 | parts | 1.5 | parts | 1.5 | parts | 1.5 | parts |

TABLE 6
Basic physical properties of Examples 5-8.

| | Example 5 | Example 6 | Example 7 | Example 8 |
|---|---|---|---|---|
| Density (g/cm³) | 0.20 | 0.20 | 0.20 | 0.20 |
| Hardness (shore C) | 38 | 38 | 38 | 38 |
| Falling ball | 68 | 68 | 68 | 68 |
| rebound rate (%) | | | | |
| 10000 repeated | 15 | 15 | 15 | 15 |
| compression | | | | |
| deformation rate (%) | | | | |

What is claimed is:

1. An antimicrobial shoe insole comprising an upper surface and a lower surface, wherein said antimicrobial shoe insole comprises an antimicrobial composition comprising at least one metal, metal-carrying agent and a binder;
wherein said metal comprises silver, silver ion, zinc, zinc ion, copper, copper ion, iron, iron ion, or a combination thereof;
wherein said metal-carrying agent comprises diatomite, diatomaceous earth, montmorillonite, silica powder, oyster shell powder, shell powder, activated carbon powder, graphite powder, zinc oxide powder, aluminum oxide powder, ferric oxide powder, or a combination thereof; and
wherein said metal and said metal-carrying agent are dispersed in said binder.

2. The antimicrobial shoe insole of claim 1, wherein said metal-carrying agent are porous and said metal is dispersed and supported in said porous metal-carrying agent and wherein said antimicrobial composition is stable at high temperature.

3. The antimicrobial shoe insole of claim 1, wherein said binder comprises hydrogenated styrene butadiene elastomer (SEBS), chemical or physical (supercritical) foaming 3.2 SEBS, a random or block copolymer SEBS, and wherein said SEBS has a molecular weight more than 100,000 Dalton, hydrogenation degree 98% or more, and a styrene content in a range of from 1% to about 40%.

4. The antimicrobial shoe insole of claim 1, wherein said antimicrobial shoe insole has a density from 0.12 to 0.30 g/cm³, preferably 0.18 to 0.22 g/cm³, a hardness of the shore C value in arrange of from 28 to 45, a ball rebound rate ≥65%, and anti-fatigue test (20% compression rate) over 10,000 times.

5. The antimicrobial shoe insole of claim 1, wherein said antimicrobial shoe insole is produced to have a sterilization rate at least 99.99%, durable for at least six months, and a mildew resistance index of zero.

6. The antimicrobial shoe insole of any one of claims 1-5 further comprising one or more pre-marked size markers on said upper surface, said lower surface, or a combination thereof.

7. The antimicrobial shoe insole of any one of claims 1-5, wherein said 10% to 100% of said antimicrobial composition are positioned on said upper surface.

8. The antimicrobial shoe insole of any one of claims 1-5, wherein said 10% to 100% of said antimicrobial composition are positioned on said lower surface.

9. The antimicrobial shoe insole of any one of claims 1-7 further comprising at least one intermediate layer positioned between said upper surface and lower surface, wherein said intermediate layer comprises moisture absorbing materials.

10. The antimicrobial shoe insole of claim 9, wherein said intermediate layer further comprises said antimicrobial composition.

11. A method for reducing odor of a shoe, said method comprising placing an antimicrobial shoe insole of any one of claims 1-10 to the inside of said shoe.

12. A shoe and shoe insole unit comprising the antimicrobial shoe insole of any one of claims 1-10.
